(19) United States
(12) Patent Application Publication
(10) Pub. No.: US 2019/0343874 A1
(43) Pub. Date: Nov. 14, 2019

(54) USE OF NICKEL COMPLEX IN PREPARING DRUG FOR RELIEVING INFLAMMATION

(71) Applicant: NATIONAL TAIWAN NORMAL UNIVERSITY, TAIPEI CITY (TW)

(72) Inventors: WAY-ZEN LEE, TAIPEI CITY (TW); CHIANG-TING CHIEN, TAIPEI CITY (TW)

(73) Assignee: NATIONAL TAIWAN NORMAL UNIVERSITY, TAIPEI CITY (TW)

(21) Appl. No.: 16/026,869

(22) Filed: Jul. 3, 2018

(30) Foreign Application Priority Data
May 10, 2018 (TW) 107115858

(51) Int. Cl. A61K 33/24; A61P 29/00

(52) U.S. Cl. CPC A61K 33/24; A61P 29/00

(57) ABSTRACT

Use of a nickel complex in preparing drugs for relieving inflammation is disclosed. The nickel complex having at least one of a structural formula (I) and a structural formula (II):

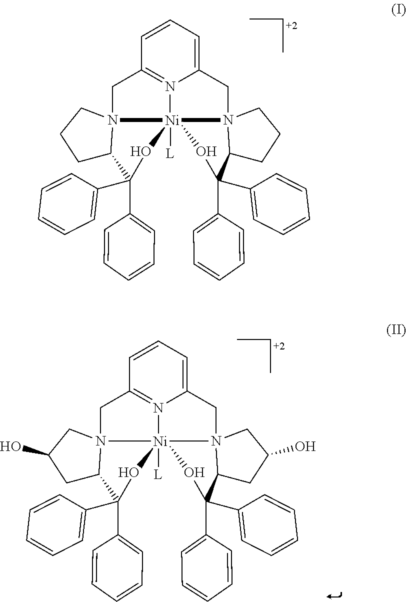

wherein, L is a solvent molecule. The nickel complex has a similar reactivity with the active site of nickel-containing superoxide dismutase (NiSOD), which could remove reactive oxygen species, such that the nickel complex could be used to prepare drugs for relieving inflammation.

### BACKGROUND OF THE INVENTION

### Technical Field

The present invention is related to a drug prepared by a nickel complex, and more particularly related to a use of a nickel complex in preparing drugs for relieving inflammation.

### Description of Related Art

Reactive oxygen species (ROS), such as superoxide anion (O₂⁻), hydrogen peroxide (H₂O₂), hypochlorous acid (HOCl) and the like, play an essential role in human's immune system which could remove bacteria and infected cells to keep people away from diseases. However, an excessive amount of reactive oxygen species would result in oxidative damage to the human body. For example, reactive oxygen species may stimulate inflammation cells and induce inflammatory response or damage blood vessels, etc.

Under normal physiological conditions, reactive oxygen species would be generated through a catalytic reaction of phagocyte with related enzymes. Besides, the chemicals existed in external environment also could induce the formation of reactive oxygen species. For example, para-phenylenediamine (PPD) is commonly used as one of the ingredients of hair dyes, and has also been used as a pigment in Henna Tattoo body art by some immoral traders. However, para-phenylenediamine is very harmful to human health since it could induce the formation of reactive oxygen species in the cells such as keratinocyte, germ cells, urothelial epithelial cells, and kidney cells, and cause bladder inflammation, bladder dysfunction, and even bladder cancer.

### BRIEF SUMMARY OF THE INVENTION

In view of the above, an object of the present invention is to provide a use of a nickel complex in preparing drugs for relieving inflammation so as to relieve inflammatory response.

According to the present invention, the use of a nickel complex in preparing drugs for relieving inflammation includes a nickel complex having at least one of a structural formula (I) and a structural formula (II):

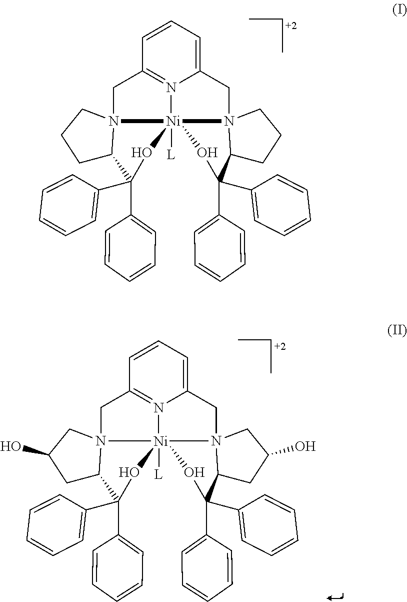

wherein, L is a solvent molecule.

The advantage of the present invention in that the nickel complex has a similar reactivity with the active site of nickel-containing superoxide dismutase (NiSOD), which could remove reactive oxygen species, such that the nickel complex could be used to prepare drugs for relieving inflammation.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is an image of tissue slices showing that para-phenylenediamine causes an infiltration of neutrophil and an increase of mast cell (as denoted by arrows) in the bladder tissues;

FIG. 2 is a vertical bar graph illustrating the mean change of perfusion unit;

FIG. 3 is a graph showing the change in intravesical pressure and arterial pressure with time;

FIG. 4 is a graph showing the change in intravesical pressure with time in the rats of the control group, the first experimental group and the second experimental group;

FIG. 5 is a graph showing the change in intravesical pressure with time in the rats of the control group, the first experimental group and the third experimental group;

FIG. 6 is a vertical bar graph illustrating the bladder intercontraction interval;

FIG. 7 is a vertical bar graph illustrating the counts of hydrogen peroxide, hypochlorous acid, and superoxide anion in the urine (in vitro) of the rats of the control group and the first experimental group;

FIG. 8 is a vertical bar graph illustrating the counts of hydrogen peroxide, hypochlorous acid, and superoxide anion in the blood (in vitro) in the rats of the control group and the first experimental group;

FIG. 9 is a graph showing the change in the count of reactive oxygen species in the bladder of each group of rats (in vivo); and

FIG. 10 is a vertical bar graph illustrating the counts of reactive oxygen species in the bladder of each group of rats (in vivo).

### DETAILED DESCRIPTION OF THE INVENTION

The following illustrative embodiments and drawings are provided to illustrate the disclosure of the present invention, these and other advantages and effects can be clearly understood by persons skilled in the art after reading the disclosure of this specification. According to an embodiment of the present invention, a nickel complex for preparing drug of relieving inflammation has at least one selected from a group consisting of the following structural formula (I) (hereinafter, referred to as WCt003), and the following structural formula (II) (hereinafter, referred to as WCt006):

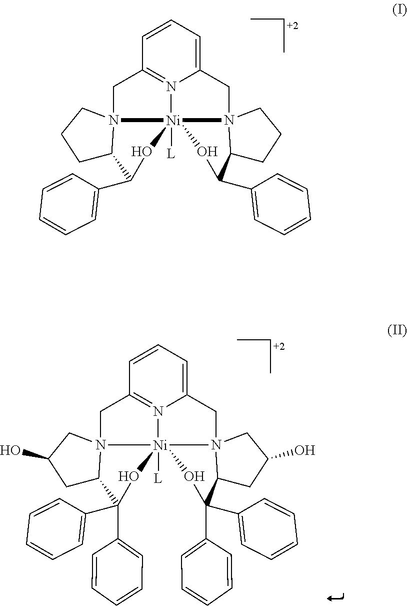

In the abovementioned structural formula, L is a solvent molecule, such as water, acetonitrile, ethanol, tert-butyl isocyanate, etc., however, this is not a limitation of the present invention.

The manufacturing method of the nickel complexes WCt003 and WCt006 has been disclosed in U.S. Pat. No. 8,642,763 and will not be described in detail herein.

Since the nickel complexes WCt003 and WCt006 respectively have a similar reactivity with the active site of nickel-containing superoxide dismutase (NiSOD), which could remove reactive oxygen species, WCt003 and WCt006 could be used to prepare drugs for relieving inflammation, especially for reducing reactive oxygen species in body fluid to relieve a bladder inflammation caused by reactive oxygen species. In the following description, several animal experiments were performed to evaluate a relieving inflammation effect of the drugs prepared from WCt003 and WCt006, wherein the animal experiments include administrating the experimental animals with para-phenylenediamine (PPD) to increase an in vivo count of reactive oxygen species, and then administrating the experimental animals with the drug prepared from WCt003 and WCt006.

In the current embodiment, 24 adult female Wistar rats were adopted as the experimental animals in the animal experiments and were randomly divided into four groups to be treated with different intraperitoneal injections. Referring to Table 1, the four groups are as follows: 1) the control group: 1 mL/day saline solution for 4 weeks; 2) the first experimental group: PPD (60 ∞g/kg/day) for 4 weeks; 3) the second group: PPD (60 g/kg/day) for 4 weeks and a drug (1.5 mg/kg/day) prepared from WCT003 for the last 2 weeks of; 4) the third experimental group: PPD (60 μg/kg/day) for 4 weeks and a drug (1.5 mg/kg/day) prepared from WCT006 for the last 2 weeks.

TABLE 1

| | | | |
|---|---|---|---|
| 1^{st} week | 2^{nd} week | 3^{rd} week | 4^{th} week |

| Control Group | normal saline | |
|---|---|---|
| First | para-phenylenediamine | |
| Experimental | | |
| Group | | |
| Second | para- | para- |
| Experimental | phenylenediamine | phenylenediamine |
| Group | | and WCt003 |
| Third | para- | para- |
| Experimental | phenylenediamine | phenylenediamine |
| Group | | and WCt006 |

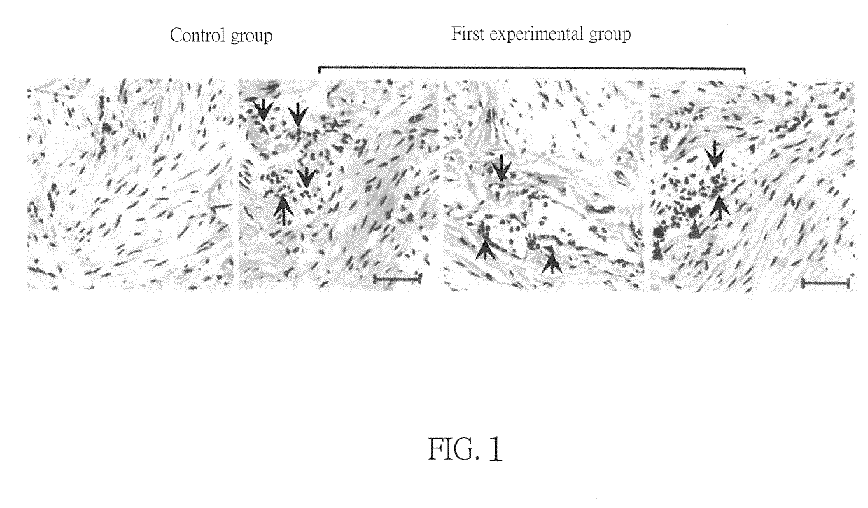

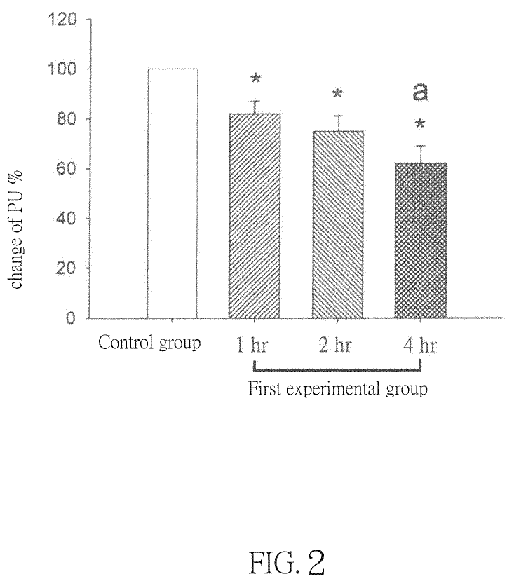

As shown in FIG. 1 and FIG. 2, as comparing the rats of the first experimental group to the rats of the control group, it is investigated that the rats of the first experimental group have neutrophil infiltration in the bladder tissue and a significant increase in the mast cell (indicated by arrow) in the bladder thereof. In addition, the perfusion unit of the rats of the first experimental group is also reduced. It could be known from the experiment data that para-phenylenediamine induces inflammatory response and ischemia in the bladders of the rats in the first experimental group.

Referring to Table 2, which lists the sample data collected from the rats of the control group and the first experimental group where were placed in the metabolic cage:

TABLE 2

| | | First |
|---|---|---|
| | | Experimental |
| | Control Group | Group |
| Body Weight (g) | 235.7 ± 16.2 | 277.8 ± 3.8 |
| Urinary frequency (24 | 33.9 ± 4.2 | 53.1 ± 13.3* |
| hours) | | |
| Water intake (ml) | 47.9 ± 8.3 | 57.7 ± 7.2* |
| Food (g) | 27.1 ± 2.8 | 22.9 ± 4.4 |
| Urine (mg) | 25.6 ± 5.1 | 32.7 ± 7.5* |
| Stool (g) | 24.3 ± 2.6 | 35.7 ± 8.1* |

*P < 0.05

It could be known from Table 2 that the urinary frequency of the rats of the first experimental group is more frequent than the rats of the control group, and the rats of the first experimental group also had more urine and stool.

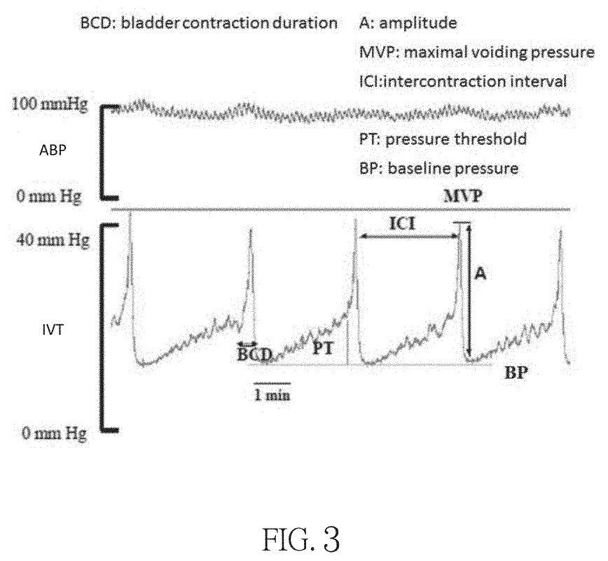

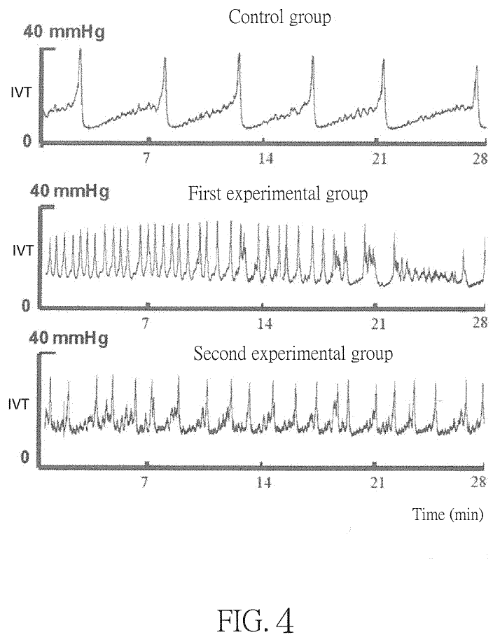

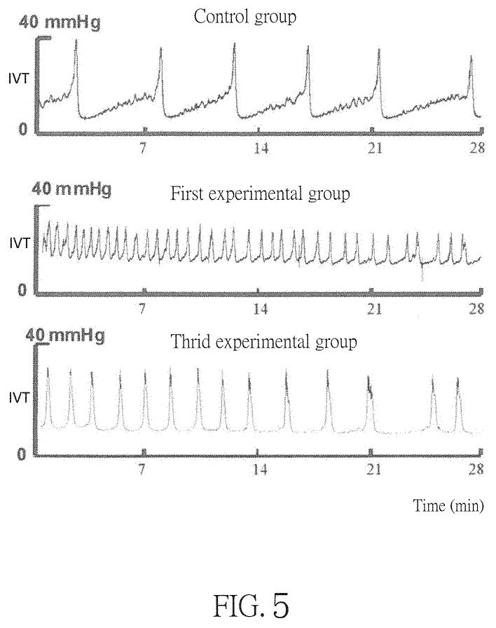

Then, each group of the rats were anesthetized with urethane (1.2 g/kg) via percutaneously injection, and a catheter was placed in a left femoral artery of each of the rats to measure arterial pressure (ABP). Meanwhile, intravesical pressure of the rats were also measured to obtain the parameters such as bladder intercontraction interval (ICI), urination time (MT), maximum urination pressure (MVP), contraction amplitude (A=MVP-BP), urination pressure threshold (PT), etc., as shown in FIG. 3 to FIG. 5.

Referring to Table 3, which lists the parameters obtained from measuring intravesical pressure of each group of the rats:

TABLE 3

| | | First | Second | Third |
|---|---|---|---|---|
| | Control | Experimental | Experimental | Experimental |
| | Group | Group | Group | Group |
| ICI (s) | 444.0 ± 62.8 | 35.8 ± 6.0* | 82.3 ± 13.6* | 179.0 ± 50.6* |
| MVP | 28.5 ± 6.5 | 30.6 ± 0.9 | 29.9 ± 2.6 | 33.1 ± 2.4 |
| (mmHg) | | | | |
| A | 17.7 ± 5.1 | 20.3 ± 0.9 | 20.1 ± 2.3 | 19.3 ± 1.8 |
| (mmHg) | | | | |

*P < 0.05

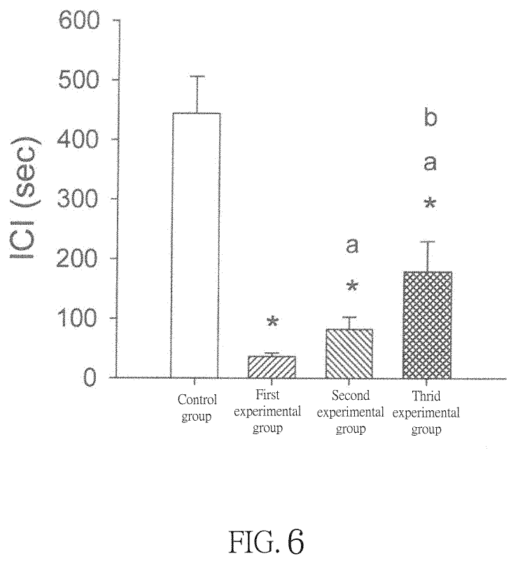

It could be known from Table 3 and FIG. 6 that rats treated with intraperitoneal injection of para-phenylenediamine (i.e., the first to the third experimental groups) had a significantly shorter bladder intercontraction interval than the rats which were not treated with intraperitoneal injection of para-phenylenediamine (i.e., the control group). Moreover, the rats treated with the drug prepared from WCt006 or WCt003 (i.e., the second and the third experimental groups) had a significantly higher bladder intercontraction interval than the rats of the first experimental group which were not treated with the drugs. It could be seen that the drugs prepared from WCt003 and WCt006 could prolong the bladder intercontraction interval and improve the voiding frequency caused by para-phenylenediamine, wherein, the therapeutic effect of the drug prepared from WCt006 is even better than that of the drug prepared from WCt003.

To determine the effect of the drug prepared from the nickel complex WCt003 and WCt006 on eliminating reactive oxygen species, a luminol and lucigenin chemiluminescence (CL) method was utilized. The method includes the following steps: 0.2 ml sample of blood or urine was mixed with 0.5 ml of 1 mmol/L lucigenin or 0.2 mmol/L luminol, and then the mixture was analyzed with a chemiluminescence analysis system, wherein, the recorded signals were corresponding to the hydrogen peroxide (H₂O₂) and hypochlorous acid (HOCl) count in the luminol method, while the recorded signals were corresponding to the superoxide anion (O₂⁻) count in the lucigenin method.

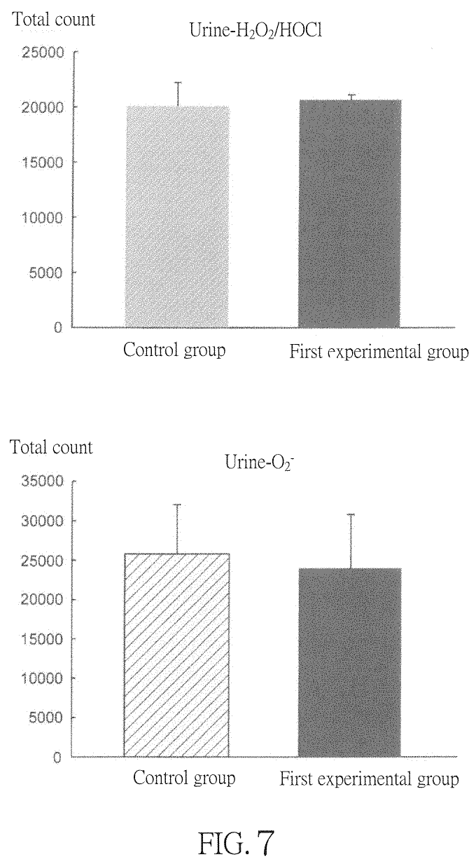

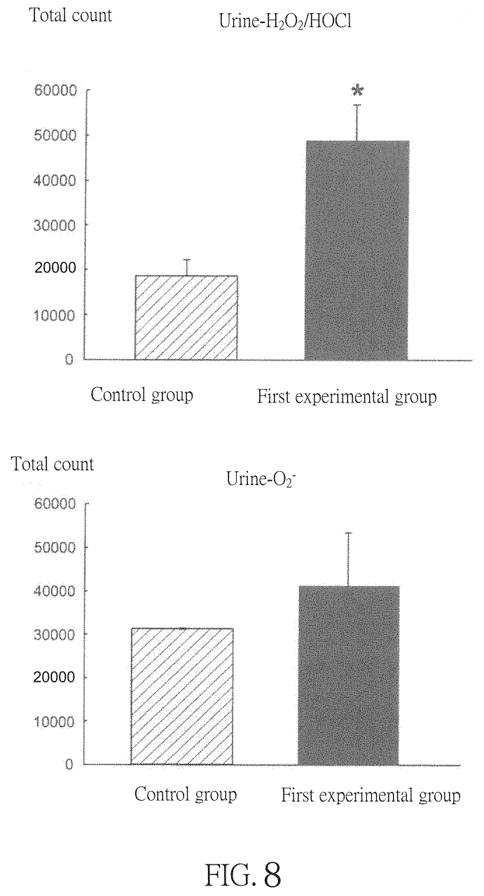

As shown in FIG. 7, there is no significant difference in the reactive oxygen species count between the urine samples of the control group and the first experimental group. However, referring to FIG. 8, the H₂O₂ and HOCl counts in the blood sample of the first experimental group is significantly higher than that of the control group; the O₂⁻ count in the blood sample of the first experimental group is also higher than that of the control group.

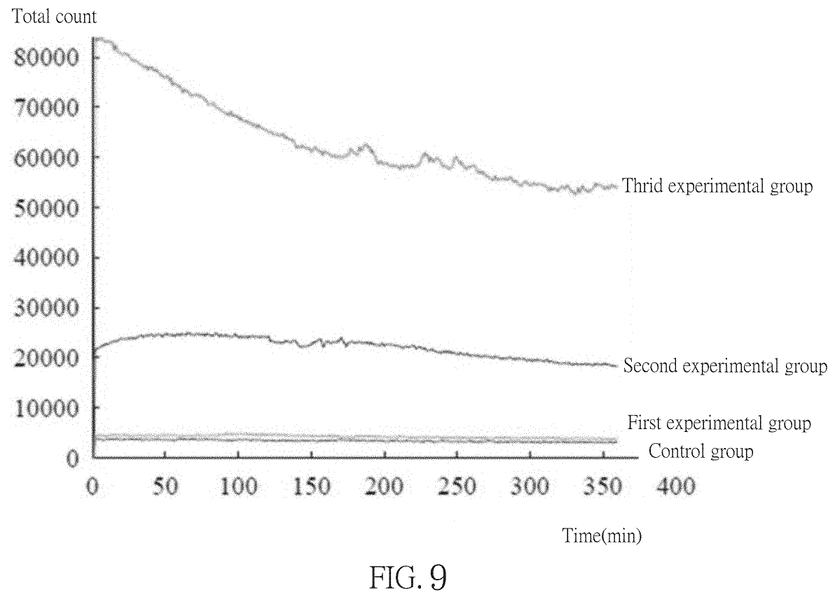

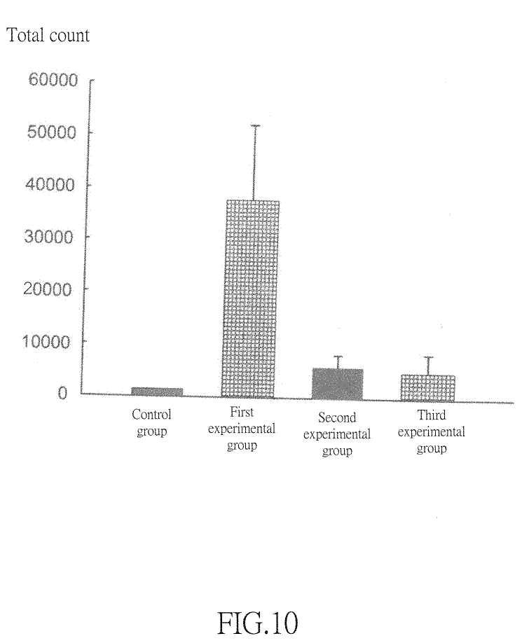

Referring to FIG. 9, which shows the detected reactive oxygen species count in vivo for each group of rats. FIG. 10 show the analytical data of the reactive oxygen species count in vivo for each group of rats. It could be seen from FIG. 9 and FIG. 10 that the reactive oxygen species count in the bladder cells of the first experimental group of rats is much higher than that of the control group. It proves that para-phenylenediamine could induce a large number of reactive oxygen species in the bladder cells, and the reactive oxygen species count could be significantly reduced again when the rats were treated with the drug prepared from WCt003 or WCt006 (respectively second experimental group and the third experimental group). Hence, the drug prepared from WCt003 or WCt006 could help to eliminate the reactive oxygen species in vivo for the rats.

From the aforementioned experimental results, it could be seen that the nickel complexes WCt003 and WCt006 could be used to prepare the drugs for relieving inflammation, which could alleviate bladder inflammation, and eliminate reactive oxygen species, such as relieving inflammatory response caused by superoxide anion (O₂⁻), hydrogen peroxide (H₂O₂), singlet oxygen (¹O₂), hypochlorous acid (HOCl), nitrogen monoxide (NO), nitrogen dioxide (NO₂), etc.

The count of WCt003 or WCt006 in the drug used in the above experiments is 1.5 mg/kg, however, this is not a limitation of the present invention. In other embodiments, the WCt003 or WCt006 count could be between 0.15 mg/kg to 15 mg/kg and the effect of relieving inflammation could be achieved. Preferably, the WCt003 or the WCt006 count could be between 0.5-2.5 mg/kg.

In the aforementioned experiments, the drugs prepared from WCt003 and WCt006 were treated via intraperitoneal injection (intraperitoneally administered), however, this is not a limitation of the present invention. Other types of treatment such as transdermal administration, oral administration bladder irrigation, etc. also could be utilized.

In summary, the nickel complexes WCt003 and WCt006 could be used to prepare the drugs for relieving inflammation, which could reduce the voiding frequency caused by bladder inflammation and eliminate the reactive oxygen species.

It must be pointed out that the embodiments described above are only some embodiments of the present invention. All equivalent structures which employ the concepts disclosed in this specification and the appended claims should fall within the scope of the present invention.

1. A method of relieving inflammation comprising
administering a nickel complex of structural formula (I) or structural formula (II), 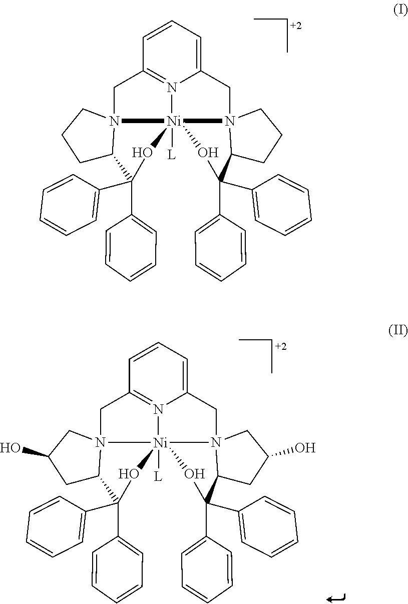
wherein, L is a solvent molecule.

2. The method according to claim 1, wherein the nickel complex is used to prepare drugs for relieving bladder inflammation.

3. The method according to claim 1, wherein the nickel complex is used to prepare drugs for relieving inflammatory response caused by reactive oxygen species.

4. The method according to claim 1, wherein the nickel complex is used to prepare drugs for reducing reactive oxygen species count in body fluids.

5. The method according to claims 3 or 4, wherein the reactive oxygen species includes at least one selected from the group consisting of superoxide anion (O₂⁻), hydrogen peroxide (H₂O₂), singlet oxygen (¹O₂), hypochlorous acid (HOCl), nitrogen monoxide (NO), and nitrogen dioxide (NO₂).

6. The method according to claims 1, 2, 3 or 4, wherein the dose of nickel complex contained in the drug is 0.15-15 mg/kg.

7. The method according to claim 6, wherein the dose of nickel complex contained in the drug is 0.5-2.5 mg/kg.

8. The method according to claim 7, wherein the dose of nickel complex contained in the drug is 1.5 mg/kg.

9. The method according to claims 1, 2, 3 or 4, wherein the drug is for intravenous injection, transdermal administration, intraperitoneally administered, oral administration or bladder irrigation.
